(19) United States
(12) Patent Application Publication
(10) Pub. No.: US 2023/0060807 A1
(43) Pub. Date: Mar. 2, 2023

(54) ANTIPERSPIRANT FORMULATIONS COMPRISING HEMISQUALANE

(71) Applicant: AMYRIS, INC., Emeryville, CA (US)

(72) Inventors: Diva CHAN, Emeryville, CA (US); RO OTERI, Emeryville, CA (US); Ashlee NUNES, Emeryville, CA (US)

(21) Appl. No.: 17/775,071

(22) PCT Filed: Nov. 6, 2020
(86) PCT No.: PCT/US20/59318

§ 371 Date: May 6, 2022

Related U.S. Application Data
(60) Provisional application No. 62/933,006, filed on Nov. 8, 2019.

(51) Int. Cl. A61K 8/31; A61Q 15/00; A61K 8/28

(52) U.S. Cl. CPC A61K 8/31; A61Q 15/00; A61K 8/28; A61K 2800/34

(57) ABSTRACT

Provided herein antiperspirant composition formulations that contain hemisqualane and lack organosilicon compounds. The hemisqualane provides the carrier and emollient activities that are typically supplied by an organosilicon compound. The antiperspirant formulations provided herein have a benefit of not releasing organosilicon compounds into the environment during and after their use.

### FIELD OF THE INVENTION

The present disclosure relates to formulations of antiperspirant compositions comprising hemisqualane and lacking any volatile silicon compounds and methods of using the antiperspirant compositions.

### BACKGROUND

Antiperspirant compositions have become part of the daily grooming routines of many people. Currently, most antiperspirant compositions contain volatile silicon compounds which serve multiple functions in the antiperspirant compositions including as a conditioning agent, solvent, humectant, carrying agent, and viscosity controlling agent. An important aspect of antiperspirant formulations is the necessity that they have significant dry feel after application. Given the widespread use of antiperspirants, there is growing concern about the environmental impact of the use of volatile silicon compounds. Studies have shown that volatile silicon compounds used in antiperspirants concentrate in the ecosystem's water after use. Alarmingly, volatile silicon compounds have been found in many forms of aquatic wildlife. Given the environmental impact of volatile silicon compounds used in antiperspirant compositions, there is a pressing need for renewable and environmentally safe replacements that still maintain a significant dry feel after application.

### SUMMARY OF THE INVENTION

The invention relates to antiperspirant formulations that contain hemisqualane. In preferred embodiments the invention relates to antiperspirant formations which contain hemisqualane in place of organosilicon compounds and which contain solid adsorbent particles.

In one aspect the invention provides for an antiperspirant formulation containing hemisqualane. In one embodiment the antiperspirant also contains one or more solid dry-feel particles. In an embodiment the solid dry-feel particle is a solid adsorbent particle. In another embodiment the solid dry-feel particle is a solid absorbent particle. In another embodiment the antiperspirant formulation is not an aerosol. In another embodiment the antiperspirant formulation does not contain or lacks an organosilicon compound. In yet another embodiment the organosilicon compound which is lacking is a cyclomethicone. In an embodiment the cyclomethicone which is lacking is cyclotetrasiloxane (D4). In an embodiment the cyclomethicone that is lacking is cyclopentasiloxane (D5). In an embodiment the cyclomethicone that is lacking is cyclohexasiloxane (D6). In further embodiments the one or more solid adsorbent particle comprises from about 0% to about 5% of the formulation. In preferred embodiments the one or more solid adsorbent particle is selected from polymer-based microspheres, allyl methacrylates crosspolymers, lauryl methacrylate/glycol dimethacrylate crosspolymer, silica, and talc. In an embodiment the antiperspirant formulation contains an active ingredient. In an embodiment the active ingredient comprises an aluminum salt. In another embodiment the active ingredient is selected from aluminum chloride; aluminum chlorohydrate; aluminum chlorodydrex; aluminum chlorohydrex propylene glycol; aluminum dichlorodhydrate; aluminum dichlorodhydrex polyethylene glycol; aluminum dichlorohydrex propylene glycol; aluminum sesquichlorohydrate; aluminum sesquichlorohydrex propylene glycol; aluminum zirconium octachlorohydrate; aluminum zirconium octachlorohydrex gly.; aluminum zirconium pentachlorohydrate; aluminum zirconium pentachlorohydrex gly; aluminum zirconium tetrachlorohydrate; aluminum zirconium tetrachlorohydrex gly; aluminum zirconium trichlorohydrate; and aluminum zirconium trichlorohydrex gly. In a preferred embodiment the active ingredient is aluminum zirconium tetrachlorohydrex glycine. In other embodiments the active ingredient is from about 0.1% to about 30% (w/w). In yet another embodiment the active ingredient is from about 10% to about 25% (w/w). In preferred embodiments the active ingredient is from about 16% to about 20%. In another embodiment the active ingredient is about 18% (w/w). In an embodiment the antiperspirant formulation contains one or more inactive ingredients. In a preferred embodiment the one or more inactive ingredients are selected from wax, emulsifier, emollient, moisturizer, anti-bacterial agent, preservative, humectant, buffer, color, and fragrance. In an embodiment the hemisqualane is from about 1% to about 80% (w/w) of the antiperspirant formulation. In yet another embodiment the hemisqualane is from about 25% to about 75% (w/w) of the antiperspirant formulation.

In another aspect the invention provides a method of preventing perspiration involving applying an antiperspirant comprising the antiperspirant formulation of the invention to an area of skin in need of application. In an embodiment, the antiperspirant is applied at least once every 24 hours. In yet another embodiment the antiperspirant is applied only once every 24 hours.

### BRIEF DESCRIPTION OF THE DRAWINGS

FIG. 1 is a spider chart showing the relative attributes of the two antiperspirant formulations immediately after application. The sample indicated 207/APAD011D5 Sep. 20, 2019 is the D5 sample whereas the sample indicated 839/APAD013HS Sep. 23, 2019 is the HS sample.

FIG. 2 is a bar graph showing the relative attributes of the two antiperspirant formulations immediately after application. The sample indicated 207/APAD011D5 Sep. 20, 2019 is the D5 sample whereas the sample indicated 839/APAD013HS Sep. 23, 2019 is the HS sample.

FIG. 3 is a spider chart showing the relative residues of the two antiperspirant formulations immediately after application. The sample indicated 207/APAD011D5 Sep. 20, 2019 is the D5 sample whereas the sample indicated 839/APAD013HS Sep. 23, 2019 is the HS sample.

FIG. 4 is a bar graph showing the relative residues of the two antiperspirant formulations immediately after application. The sample indicated 207/APAD011D5 Sep. 20, 2019 is the D5 sample whereas the sample indicated 839/APAD013HS Sep. 23, 2019 is the HS sample.

FIG. 5 is a spider chart showing the relative attributes of the two antiperspirant formulations 5-minutes after application. The sample indicated 207/APAD011D5 Sep. 20, 2019 is the D5 sample whereas the sample indicated 839/APAD013HS Sep. 23, 2019 is the HS sample.

FIG. 6 is a bar graph showing the relative attributes of the two antiperspirant formulations 5-minutes after application. The sample indicated 207/APAD011D5 Sep. 20, 2019 is the D5 sample whereas the sample indicated 839/APAD013HS Sep. 23, 2019 is the HS sample.

FIG. 7 is a spider chart showing the relative residues of the two antiperspirant formulations 5-minutes after application. The sample indicated 207/APAD011D5 Sep. 20, 2019 is the D5 sample whereas the sample indicated 839/APAD013HS Sep. 23, 2019 is the HS sample.

FIG. 8 is a bar graph showing the relative residues of the two antiperspirant formulations 5-minutes after application. The sample indicated 207/APAD011D5 Sep. 20, 2019 is the D5 sample whereas the sample indicated 839/APAD013HS Sep. 23, 2019 is the HS sample.

FIG. 9 is a spider chart showing the relative attributes of the two antiperspirant formulations 15-minutes after application. The sample indicated 207/APAD011D5 Sep. 20, 2019 is the D5 sample whereas the sample indicated 839/APAD013HS Sep. 23, 2019 is the HS sample.

FIG. 10 is a bar graph showing the relative attributes of the two antiperspirant formulations 15-minutes after application. The sample indicated 207/APAD011D5 Sep. 20, 2019 is the D5 sample whereas the sample indicated 839/APAD013HS Sep. 23, 2019 is the HS sample.

FIG. 11 is a spider chart showing the relative residues of the two antiperspirant formulations 15-minutes after application. The sample indicated 207/APAD011D5 Sep. 20, 2019 is the D5 sample whereas the sample indicated 839/APAD013HS Sep. 23, 2019 is the HS sample.

FIG. 12 is a bar graph showing the relative residues of the two antiperspirant formulations 15-minutes after application. The sample indicated 207/APAD011D5 Sep. 20, 2019 is the D5 sample whereas the sample indicated 839/APAD013HS Sep. 23, 2019 is the HS sample.

FIG. 13 is a bar graph comparing the subject satisfaction between an HS sample antiperspirant and a D5 sample antiperspirant plotting percent satisfaction for each of fifteen distinct aspects associated with antiperspirant use.

### DETAILED DESCRIPTION OF THE EMBODIMENTS

As used herein “active ingredient” or “active antiperspirant ingredient” means the ingredient that causes the reduction in sweat production after application of the antiperspirant composition to a subject. The active ingredient may comprise aluminum-containing salts including aluminum-zirconium salts. A non-limiting list of active ingredients includes aluminum chloride; aluminum chlorohydrate; aluminum chlorodydrex; aluminum chlorohydrex propylene glycol; aluminum dichlorodhydrate; aluminum dichlorodhydrex polyethylene glycol; aluminum dichlorohydrex propylene glycol; aluminum sesquichlorohydrate; aluminum sesquichlorohydrex propylene glycol; aluminum zirconium octachlorohydrate; aluminum zirconium octachlorohydrex gly; aluminum zirconium pentachlorohydrate; aluminum zirconium pentachlorohydrex gly; aluminum zirconium tetrachlorohydrate; aluminum zirconium tetrachlorohydrex gly; aluminum zirconium trichlorohydrate; and aluminum zirconium trichlorohydrex gly.

As used herein, “subject” is an organism that is tested with one or more compositions of the invention. In some embodiment, the subject is a mammalian subject, such as a human or a domestic animal.

As used herein, the term “about” will be understood by persons of ordinary skill in the art and will vary to some extent on the context in which is used. If there are uses of the term which are not clear to persons of ordinary skill in the art given the context in which is used, ‘about’ may mean up to plus or minus 20% of the particular term.

As used herein, the term “ointment” may be any commonly known and commercially available ointments.

As used herein “hemisqualane” or “farnesane” refers to a compound having the following structure:

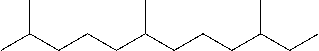

or a stereoisomer thereof.

As used herein “organosilicon compound” refers to an organometallic compound containing carbon—silicon bonds. A particular class of examples of an organosilicon compounds are the cyclomethicones which are volatile organosilicon compounds that are often used in antiperspirants formulations. Illustrative examples of organosilicon compounds include cyclotetrasiloxane (D4), cyclopentasiloxane (D5), cyclohexasiloxane (D6).

As used herein “antibacterial ingredient” or “antibacterial” means a compound or agent that works to eliminate the bacteria that cause bad odors from areas where sweat is common.

As used herein “inactive ingredients” are compounds that do not directly act to reduce sweat formation in an antiperspirant but function in one or more secondary roles that provide desirable qualities to the antiperspirant. Non-limiting illustrative examples of inactive ingredient classes includes emollients, moisturizers (e.g. mineral oil), anti-bacterial ingredients, preservatives (e.g. sodium benzoate; parabens, BHT), humectants (e.g. propylene glycol; glycerine), buffers (pH control agents), colors, and fragrances.

As used herein “solid dry-feel particle” refers to materials that when used in antiperspirant compositions reduce the greasy or moist feeling or sensation of the formulations when applied. Two distinct classes of solid dry-feel particle exist: solid adsorbent particles and solid absorbent particles which operate by adsorption or absorption respectively. Non-limiting examples of solid adsorbent particles include polymer-based microspheres (e.g. MICROSPONGE), allyl methacrylates crosspolymers (e.g. POLY-PORE), lauryl methacrylate/glycol dimethacrylate crosspolymer (e.g. POLYTRAP), silica, and talc. In preferred embodiments of the invention, solid adsorbent particles comprise from about 0% to about 5% of the composition.

In some embodiments, the compositions of the present invention are free of glycine, other than glycine that is complexed with the active antiperspirant ingredient. The compositions may further be free of calcium salts such as calcium chloride and calcium carbonate. Gel filtration may be used to remove glycine and calcium salts from the composition. Other suitable methods of removing glycine and calcium salts would be known to the skilled person. Removal of calcium and/or glycine enhances the efficacy of the antiperspirant active and allows a smaller amount to be used in the compositions.

The active antiperspirant ingredient can be incorporated into the compositions of the present invention in an amount of 5 weight % to 20 weight % (on an anhydrous basis) by total weight of the composition. In some embodiments, the active antiperspirant ingredient is incorporated into the compositions in an amount of 5 weight % to 20 weight %, 7 weight % to 15 weight %, 10 weight % to 15 weight % or 12 weight % to 15 weight % on an anhydrous basis, by total weight of the composition.

The compositions of the present invention may further comprise one or more ingredients selected from: a deodorant active, an emollient, a surfactant, a gelling agent, a fragrance and an emulsifier. These ingredients, and the amounts in which they may be incorporated into the compositions, would be known to those of ordinary skill in the art. Specific examples are described below.

The compositions may comprise one more agents that decrease odor or odor formation. Any known deodorant can be used. Examples of deodorants include, but are not limited to antibacterials, alcohols, 2,4,4′-trichloro-2′-hydroxy diphenyl ether (Triclosan), octoxyglycerin, benzethonium chloride, polyhexamethylene biguanides, triethylcitrate, 2-amino-2-methyl-1-propanol (AMP), cetyl-trimethylammonium bromide, cetyl pyridinium chloride, bactericides, and bacteriostats.

The compositions of the present invention may further comprise one or more surfactants. The surfactant can be included in any desired amount. In one embodiment, the amount of surfactant is 2 to 12 weight % by total weight of the composition. The amount in the composition is based on the as supplied material. In another embodiment, the amount of surfactant is 3 to 10 weight % by weight. In one embodiment, when the composition is an oil-in-water roll-on formula, the amount of surfactant is from 2 to 5 weight % by total weight of the composition. Examples of the surfactant include, but are not limited to, nonionic surfactants, silicone surfactants, and combinations thereof.

Nonionic surfactants that can be used include, but are not limited to, (a) sorbitan esters and ethoxylated sorbitan esters (for example PEG-20 sorbitan isostearate, sorbitan monolaurate, polysorbate-20, polysorbate-40, polysorbate-60, polysorbate-80); (b) ethoxylates (for example, Ceteth-20, PEG-30 castor oil. PEG-40 hydrogenated castor oil, PEG-60 hydrogenated castor oil. Laureth-7, Isolaureth-6, Steareth-10. Steareth-20. Steareth-21, Steareth-100. Ceteareth-12, Oleth-5, Oleth-10); (c) ethoxylated adducts (for example, PEG-25 stearate, glyceryl stearate and PEG-100 stearate): (d) PEG esters (for example, PEG-8 oleate, PEG-8 laurate, PEG-8 dilaurate. PEG-12 dilaurate, PEG-80 diisostearate, PEG-40 stearate); (e) propoxylates (for example, PPG-10 butanediol, PPG-50 oleyl ether, PPG-2-ceteareth-9, PPG-3-deceth-3. PPG-5-ceteth-20); (f) ethoxylated modified triglycerides (for example, PEG-20 corn glycerides. PEG-12 palm kernel glycerides); (g) alkylphenol aromatic ethoxylates (for example, dinonylphenol ethoxylate with 9 moles of EO, octylphenol ethoxylate with 20 moles of EO, octylphenol ethoxylate with 40 moles of EO): (h) block copolymers that are alkoxylated glycols having ethoxylated and propoxylated segments (for example, POLOXAMER.TM. 182 and 234. POLOXAMER.TM. 105 Benzoate, and MEROXAPOL.TM. 174); and combinations thereof. In one embodiment, the nonionic surfactant is selected so that it has an HLB (hydrophilic-lipophilic balance) value of 8-16 (more particularly 8-12).

In one embodiment, the nonionic surfactant is selected from ethoxylated nonionic surfactants and propoxylated non-ionic surfactants. Example of these include, but are not limited to, Steareth 2, Steareth 20, and Steareth 21. In an oil in water composition embodiment, a combination of two surfactants, one having an HLB value of 2 to 8 (such as Steareth 2) and the other having an HLB of 9 to 18 (such as Steareth 20 and 21), can be used.

Gelling agents may further be incorporated into the compositions. Examples of gelling agents include, but are not limited to, waxes, esters of fatty acid and fatty alcohol, triglycerides, partially or fully hydrogenated soybean oil, partially or fully hydrogenated castor oil, other partial or fully hydrogenated plant oils, stearyl alcohol, or other cosmetically acceptable materials, which are solid or semi-solid at room temperature and provide a consistency suitable for application to the skin.

In one embodiment, the gelling agent comprises a combination of hydrogenated soybean oil and a hydrocarbon of the formula C.sub.nH.sub.2n+2, wherein n is 20 to 100, and the hydrocarbon is at least 90% linear. In this embodiment, the antiperspirant composition has a structure that provides a better delivery of the antiperspirant to the skin.

In certain embodiments, the fully or partially hydrogenated soybean oils are those described in US2008/0187504A1 and US2008/0187503A1. The hydrogenated soybean oil from US2008/0187504A1 is almost, but not fully hydrogenated. The amount of hydrogenation is measured by the iodine value. The iodine value can be measured by ASTM D5554-95 (2006). The iodine value of the hydrogenated soybean oil used herein is greater than 0 to 20. In one embodiment, the iodine value is 1 to 5. The partially hydrogenated soybean oil from US2008/0187503A1 has a melting point that of −15.degree. C. (5′ F) to 38.degree. C. (100.degree. F.). In another embodiment, the melting point is 26.degree. C. (80.degree. F.) to 38.degree. C. (100.degree. F.). To obtain the desired melting point, the oil can be partially hydrogenated or a blend of non-hydrogenated with partially or fully hydrogenated oils and/or waxes.

The partially or fully hydrogenated soybean oil is present in an amount up to 20% by weight of the composition. In another embodiment, the amount is up to 10% by weight. In one embodiment, the amount is at least 1, 2, 3, 4, 5, 6, 7, 8, or 9% by weight. In another embodiment, the amount is less than 10, 9, 8, 7, 6, 5, 4, 3, 2, 1% by weight. Any of the preceding minimum and maximum amounts can be combined to form any range of values.

The hydrocarbon is a hydrocarbon of the formula C.sub.nH.sub.2n+2, wherein n is 20-100, and the hydrocarbon is at least 90% linear. In one embodiment, the hydrocarbon is a paraffin. In another embodiment, the hydrocarbon is polyethylene/polymethylene. An example of a polyethylene can be found in U.S. Pat. No. 6,503,491. In another embodiment, the polyethylene has a weight average molecular weight of 300 to 3000 and a melting point of 50 to 129° C.

When water is present, for example in a liquid roll-on composition, the amount of water in the composition is the amount to make a 100% by weight composition after all of the materials, including any optional materials, are added to the composition. In certain embodiments, the amount of water is at least 20%, 30%, 40%, 50%, 60%, 70%, 80%, or 85% by weight of the composition.

The total amount of solids in the composition is the amount of non-volatile materials in the composition. The percentage of solids is measured by a CEM Smart System moisture/solids analyzer which uses microwave energy to dry the samples. In one embodiment, the total amount of solids is less than 25%. In another embodiment, the total amount of solids is less than 20%.

The compositions of the present invention may also contain particulates which include but are not limited to talc, mica, fragrance encapsulates, or hydrophobically modified starches, such as aluminum starch octenyl succinate (MACKADERM.TM. ASTRO-DRY.TM. from McIntyre Group Ltd.). If the composition is in a liquid form and dispensed through a roll-on applicator, the average particle size of the suspended material is such that the particles can pass through the application to prevent the ball applicator from malfunctioning. Usually, the average particle size does not exceed 150 microns.

Additional components of the antiperspirant compositions optionally include any components suitable for use in such compositions which are known in the art. Such components include additional preservative agents such as quaternary ammonium compounds (for example, 2-amino-2-methyl-1-propanol (AMP), cetyl-trimethylammonium bromide, cetyl pyridinium chloride, 2, 4, 5 N-trichloro-2N-hyroxydiphenylether (Triclosan) and various zinc salts), colorants, emulsifiers etc. In some embodiments, the compositions of the present invention are free of preservatives other than disodium EDTA. Antioxidants may also be incorporated into the composition, preferably to act as ingredient protectants and for maintenance of long-term stability of the composition. Suitable antioxidants include Tinogard, manufactured by Ciba Specialty Chemicals, Basel, Switzerland.

Waxes employed herein as thickening agents are often selected from hydrocarbons, linear fatty alcohols, silicone polymers, esters of fatty acids or mixtures containing such compounds along with a minority (less than 50% w/w and often less than 20% w/w) of other compounds. Naturally occurring waxes are often mixtures of compounds which include a substantial proportion of fatty esters.

Examples of hydrocarbon waxes include paraffin wax, ozakerite, microcrystalline wax and polyethylene wax, the last named desirably having an average molecular weight of from 300 to 600 and advantageously from 350 to 525.

Linear fatty alcohols commonly contain from 14 to 40 carbon atoms and often from 16 to 24. Preferred thickening agents of this class are stearyl alcohol and behenyl alcohol, with stearyl alcohol being especially preferred.

Examples of ester waxes include esters of C₁₆-C₂₂ fatty acids with glycerol or ethylene glycol, which can be isolated from natural products or more conveniently synthesised from the respective aliphatic alcohol and carboxylic acid.

Examples of natural waxes include beeswax, wool wax and spermaceti wax of animal origin, and caster wax, jojoba wax, carnauba wax and candelilla wax which are of vegetable origin. Montan wax, which is an example of mineral wax, includes non-glyceride esters of carboxylic acids, hydrocarbons and other constituents.

### EXAMPLES

### Example 1: Comparing the Properties of Cyclopentasiloxane (D5) and Hemisqualane Containing Antiperspirant

To determine if hemisqualane could act as a replacement for organosilicon compounds in antiperspirant formulations, an antiperspirant formulations containing the volatile silicon cyclopentasiloxane (D5) and hemisqualane were prepared and compared. Both formulations contained the following: Active Ingredient: Aluminum Zirconium Tetrachlorohydrex GLY—18%; Inactive Ingredients: C13-15 Alkane, Stearyl Alcohol, C12-15 Alkyl Benzoate, PPG-3 Benzyl Ether Myristate, Hydrogenated Castor Oil, Talc, Polyethylene, PEG-8 Distearate. The antiperspirant containing cyclopentasiloxane is referred to as the “D5 sample” or merely “D5” and the Hemisqualane containing antiperspirant is referred to as the “HS sample” or merely “HS.”

The two antiperspirant formulations were compared using a quantitative descriptive analysis known as the Spectrum Descriptive Analysis Method. The Spectrum Descriptive Analysis Method grounds itself in the use of published and internal intensity reference scales to define intensity boundaries in sensory experiences. A group of test subjects, the Skinfeel Panelists, were trained using the Spectrum Descriptive Analysis Method for personal care products. They were selected for their ability to detect and discriminate differences in visual and tactile properties. Panelists were trained on a universal scale that focuses on intensity or strength of the signal coupled with detailed description and definitions of sensory attributes and use of calibrated training samples. All panelists received a minimum of 100 hours of training and practice prior the experimental protocol and were extensively trained in evaluation of lotions, creams, gels, cleansers, and related product forms.

All data was collected from the individual subjects, and samples were replicated. collection of this type is well suited to correlation with both instrumental and consumer research data.

Twelve (12) subjects, trained in Spectrum Skinfeel Descriptive Analysis method, completed the study. Sample presentation was rotated among the subjects, with each sample evaluated twice. A Standard Antiperspirant-Deodorant testing protocol was used. Data was collected using a Standard Antiperspirant-Deodorant descriptive analysis ballot. Attributes were scaled using an intensity scale from none to very strong. The data was summarized for each sample and each attribute. Statistical procedures (ANOVA) were used to highlight significant differences among samples for each.

ANOVA allows the determination of significant differences among samples and for each attribute tested. The Model Statement comprises sample as a main effect; judge and replication are used as blocking factors. In the output, significant differences among samples are reported as follows: **=Significantly different at the 95% confidence level (CL). Fisher's Least Significant Difference (1sd) is used as a means separation procedure to identify where differences reside with in the set of samples tested. 1sd is reported at the 95% CL where p-value<0.05. Data was reported in a tabular format as means for scaled attributes and frequencies for check-all-that-apply attributes unless otherwise noted. Within each attribute, means that share a common letter are not statistically significantly different at the 95% confidence level. See Table 1.

The following protocol was used during the comparative testing. Subjects used a non-abrasive soap the morning of the evaluation. No lotion was applied to arms. Upon arrival at the study site, subjects washed the test site with a non-abrasive soap (Cetaphil) at least 10 minutes prior to the start of the study. Subjects used a skin scribe to make a 6 in.×2 in. rectangle in the crook of each arm insuring that the fold of the arm bisects the rectangle. Subjects marked off 4 sections, 2 on either side of the fold, leaving a 2 in. square around the fold.

The arm of each subject was held out at a right angle to the body with the back of the hand perpendicular to the floor. A technician applied the test sample by stroking in a zigzag pattern to evenly cover the surface area of the rectangle. The technician counted the number of strokes to achieve complete coverage and recorded it on the ballot. This was done to the beat of a metronome (120 bpm). The technician then weighed the sample and gave the weight measurement to the subject for recording on the ballot.

Immediately after application, the subjects assessed: Number of Strokes (how many strokes it took to cover evaluation site?); Coolness (the degree to which the sample feels “cool” on the skin); and Occlusion (the degree to which the sample occludes or blocks air passage over the skin). The subjects also assessed at that time and at 5 minutes and at 15 minutes after application: Gloss (the amount of reflected light from the skin); Visual Residue (is there product visible on the skin?); White Residue (the amount of white residue visible on the skin); Wetness (the degree to which the skin feels wet); Stickiness (the degree to which finger adheres to residual product); Slipperiness (the ease of moving fingers across the skin); Thickness of residue (the thickness of residue perceived on the skin); Amount of residue (the total amount of product still on the skin); Type of residue (Oil, Wax, Grease, Silicone, Chalky, Plastic); Flaking/Pilling (is there flaking/pilling visible on the skin?); and Stickiness (degree to which arm sticks to itself). At the 15 minute after application time point only, the subjects also assessed: Tautness (the degree to which the skin feels taut or tight and Rub-off whitening (the amount of white residue on the dark fabric).

Baseline information, when collected, reflects the condition of the skin without any product application; such information served as an internal reference for the subjects and to provide context to changes in attribute intensities caused by use of the test samples. In many cases, baseline data was not collected but is used as an internal calibration tool for panelists. Knowledge of baseline data can be helpful in interpreting differences in skin feeling for attributes such as gloss, slipperiness, etc. over the use cycle of the product evaluation and for understanding that the baseline of a given attribute may be zero or a positive value.

During application there are no significant differences between the D5 sample and HS sample for cooling and occlusion. After application, the HS sample was statistically thicker and glossier than the D5 sample across all timepoints. The HS sample felt slightly wetter (moist) immediately and 5-minutes after application, then returns to baseline at the 15-minute timepoint. Whitening on fabric (Rub-off Whitening @15 minutes) was higher for the D5 sample. The antiperspirant/deodorants did not leave much residue on skin. The intensity was low at all 3 timepoints and differences were statistically significantly different 5-minutes after application only. The character of the residue, albeit at very low intensity, was initially more oily, greasy & silicone-like in the HS sample while the D5 sample felt more powdery/chalky. The sensation changed to mainly greasy in the HS sample at the 5- and 15-minute timepoints, while the D5 sample continued to feel more powdery/chalky.

Overall, differences between the two antiperspirant/deodorants were small and attribute intensities were low. Therefore, these distinctions are not likely to be relevant or noticeable by consumers in a finished product.

### Results: Application & Immediate After-Feel

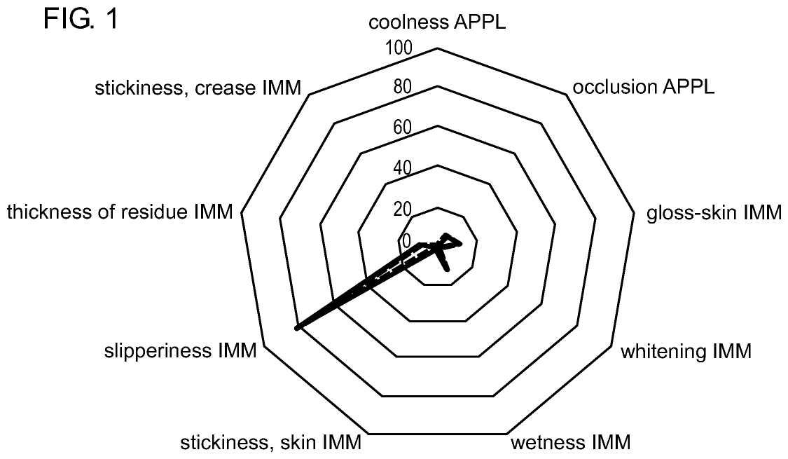

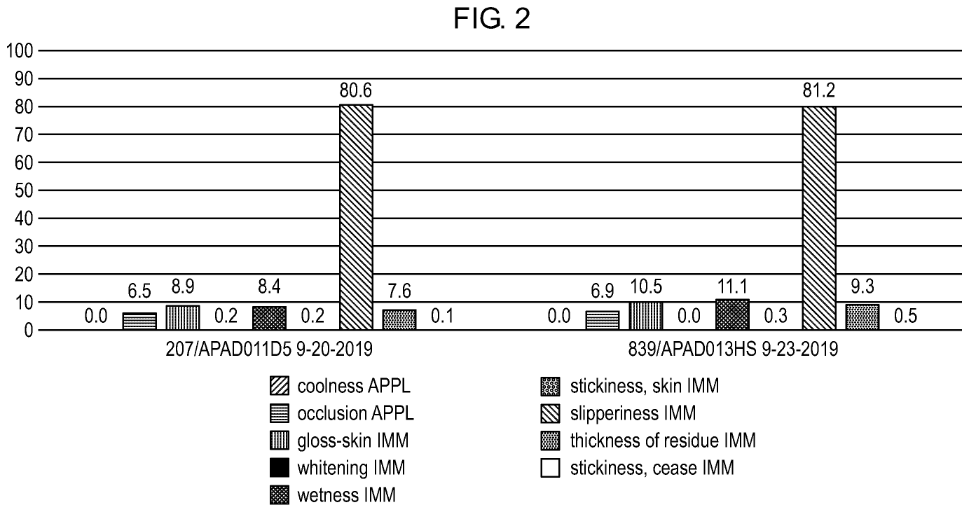

The responses during and immediately after application for the after-feel are shown in FIGS. 1 and 2. During Application there were no significant differences in cooling or occlusion. Immediately after application, the HS sample created statistically significantly higher gloss on skin, and felt wetter, thicker and more slippery than the D5 sample. Stickiness in the crease of the arm for the HS sample slightly lower. However, these differences may not be relevant because the ranges are small and attribute intensities are low.

Data analysis notes:

### Results: Immediate Residue

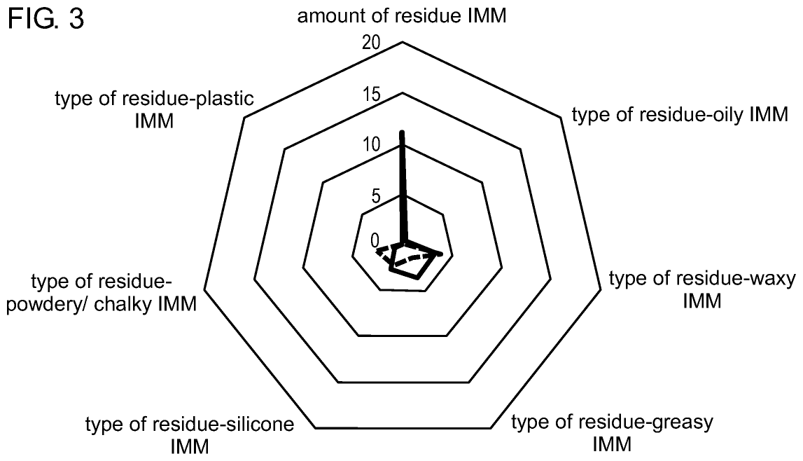

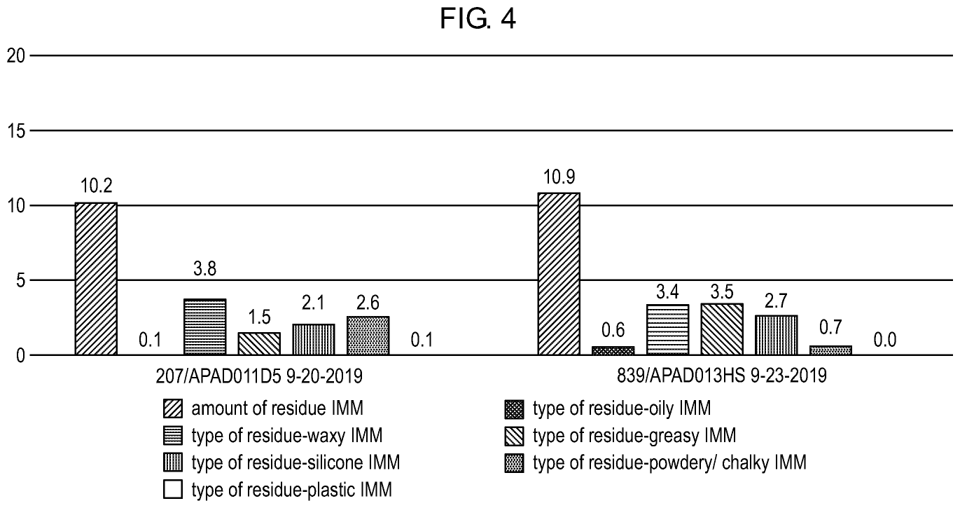

The immediate Residue data are shown in FIGS. 3 and 4. While there are no statistically significant differences between the two samples for Amount of Residue immediately after application, differences in the type of residue did surface: the HS sample was more oily, greasy and silicone-like whereas the D5 sample was more powdery/chalky and directionally more plastic-feeling. However, the differences are not likely relevant because attribute intensities were low.

### Results: 5-Minute After-Feel

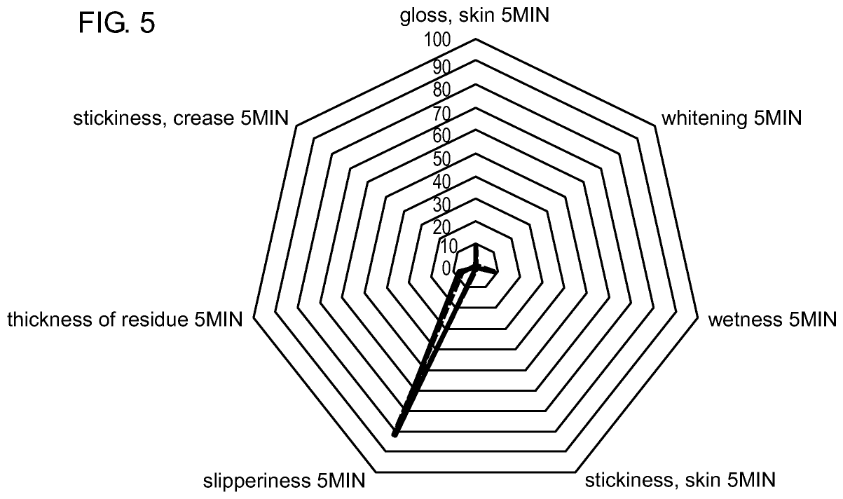

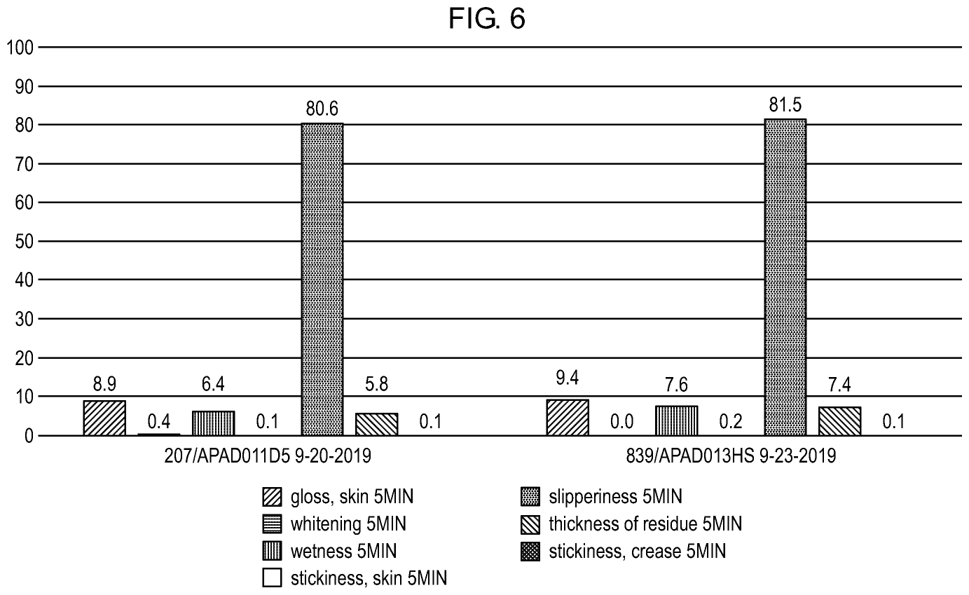

The reported results for the five-minute time point for after-feel are shown in FIGS. 5 and 6. Five minutes after application, the HS sample displayed a statistically significantly higher gloss on skin and felt wetter and thicker than the D5. However, these differences are slight and the overall after-feel between the two samples is remarkably similar.

### Results: 5-Minute Residue

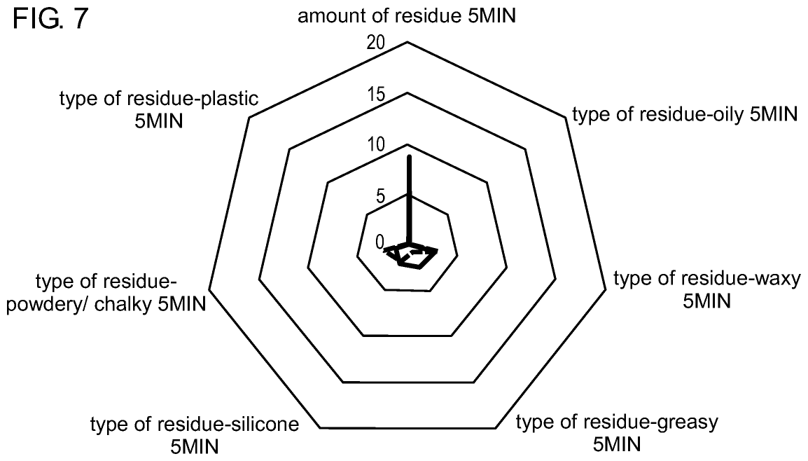

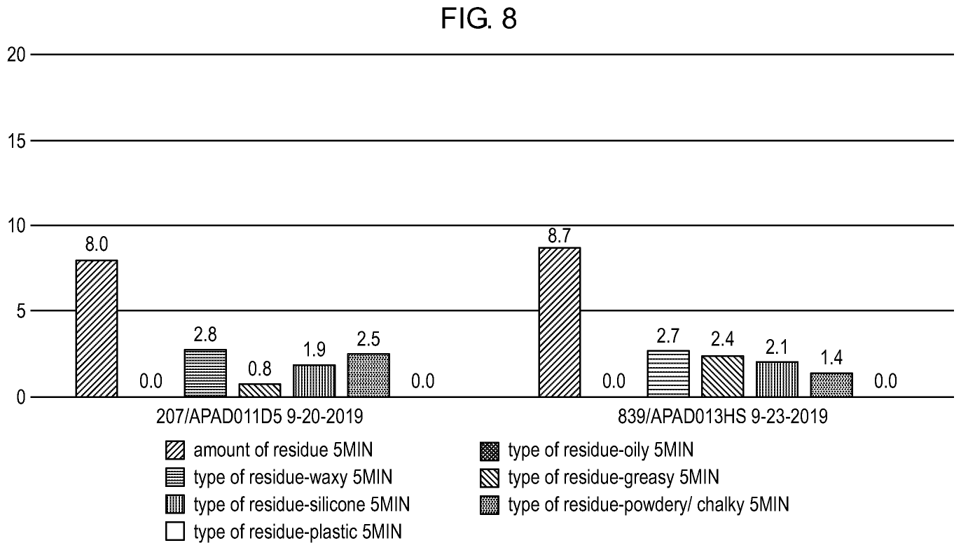

The data for the amount of residue remaining at the 5-minute point are shown in FIGS. 7 and 8. The Amount of Residue left by the HS sample five minutes after application was directionally higher than the D5 sample. The character of this residue was more greasy and less powdery/chalky.

### Results: 15-Minute After-Feel

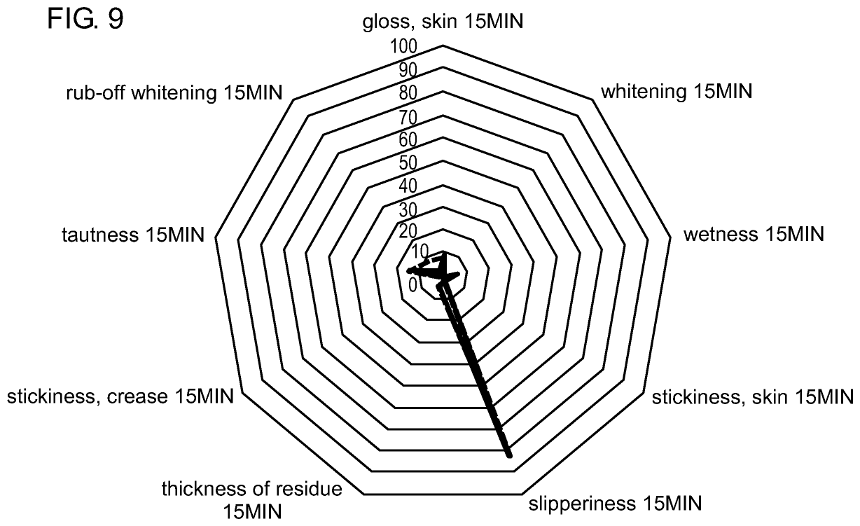

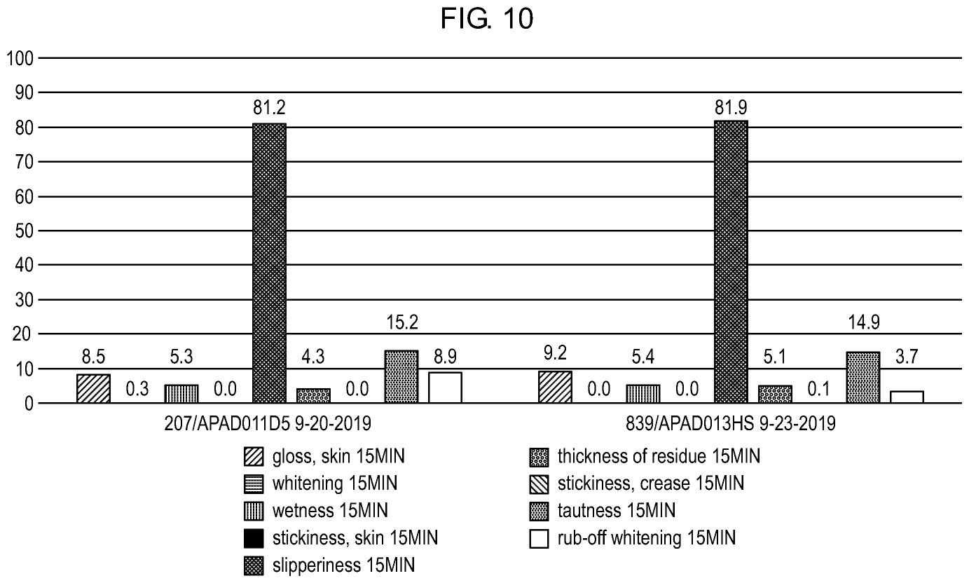

The data for the 15-minute after-feel are shown in FIGS. 9 and 10. Fifteen minutes after application, the HS sample remained statistically significantly higher in thickness and gloss on skin. It was also directionally more slippery than the D5 sample but feels less taut. Whitening on skin and fabric (Rub-off Whitening) was lower in the D5 sample. However, differences between the two antiperspirant/deodorants is not likely relevant to a consumer product because ranges are small and attribute intensities are low. Moreover, the similarities in the after-feel out weigh the differences detected.

### Results: 15-Minute Residue

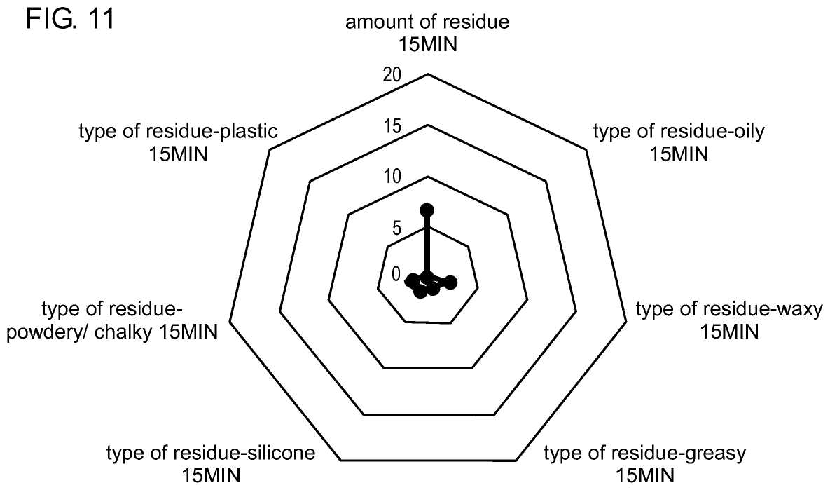

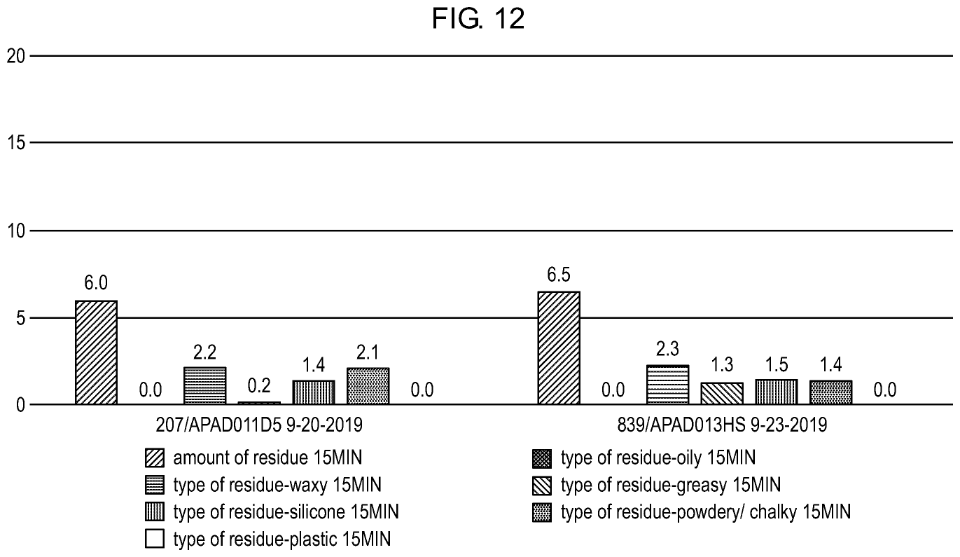

The data showing the amount of residue for the 15-minute time point are shown in FIGS. 11 and 12. While there are no statistically significant differences between samples for Amount of Residue 15 minutes after application, differences in the character of the residue remain: the HS sample was more greasy and the D5 sample was more powdery/chalky. However, differences between the two antiperspirant/deodorants may not be relevant since attribute intensities are low.

TABLE 1

| | D5 | Hemisqualane | p- | | |
|---|---|---|---|---|---|
| Attributes | Antiperspirant | Antiperspirant | value | lsd | sig |
| # strokes to cover site | 8.0 | 8.0 | — | — | |
| amount used (weight in grams) | 0.1 | 0.1 | 0.1341 | — | |
| coolness APPL | 0.0 | 0.0 | — | — | |
| occlusion APPL | 6.5 | 6.9 | 0.3964 | — | |
| gloss-skin IMM | 8.9 B | 10.5 A | 0.0013 | 0.92 | ** |
| whitening IMM | 0.2 | 0.0 | 0.3259 | — | |
| wetness IMM | 8.4 B | 11.1 A | <.0001 | 1.19 | ** |
| stickiness, skin IMM | 0.2 | 0.3 | 0.4535 | — | |
| slipperiness IMM | 80.6 B | 81.2 A | 0.0226 | 0.55 | ** |
| thicknes of residue IMM | 7.6 B | 9.3 A | 0.0025 | 1.05 | ** |
| amount of residue IMM | 10.2 | 10.9 | 0.1920 | — | |
| type of residue %-oily IMM | 0.8 B | 5.3 A | 0.0018 | 2.67 | ** |
| type of residue % waxy IMM | 36.8 | 32.0 | 0.1837 | — | |
| type of residue %-greasy IMM | 15.0 B | 31.0 A | <.0001 | 4.76 | ** |
| type of residue %-silicone IMM | 20.0 B | 24.5 A | 0.0120 | 3.43 | ** |
| type of residue %- | 26.5 A | 7.3 B | <.0001 | 5.62 | ** |
| powdery/chalky IMM | | | | | |
| type of residue %-plastic IMM | 1.0 a | 0.0 b | 0.0887 | 0.96 | * |
| type of residue-oily IMM | 0.1 B | 0.6 A | 0.0029 | 0.32 | ** |
| type of residue-waxy IMM | 3.8 | 3.4 | 0.3107 | — | |
| type of residue-greasy IMM | 1.5 B | 3.5 A | <.0001 | 0.81 | ** |
| type of residue-silicone IMM | 2.1 B | 2.7 A | 0.0188 | 0.54 | ** |
| type of residue- | 2.6 A | 0.7 B | <.0001 | 0.53 | ** |
| powdery/chalky IMM | | | | | |
| type of residue-plastic IMM | 0.1 a | 0.0 b | 0.0887 | 0.11 | * |
| stickiness, crease IMM | 0.1 b | 0.5 a | 0.0952 | 0.34 | * |
| gloss, skin 5 MIN | 8.9 B | 9.4 A | 0.0172 | 0.44 | ** |
| whitening 5 MIN | 0.4 | 0.0 | 0.1085 | — | |
| wetness 5 MIN | 6.4 B | 7.6 A | 0.0076 | 0.85 | ** |
| stickiness, skin 5 MIN | 0.1 | 0.2 | 0.1780 | — | |
| slipperiness 5 MIN | 80.6 | 81.5 | 0.1048 | — | |
| thickness of residue 5 MIN | 5.8 B | 7.4 A | 0.0021 | 0.97 | ** |
| amount of residue 5 MIN | 8.0 b | 8.7 a | 0.0835 | 0.66 | * |
| type of residue %-oily 5 MIN | 0.0 | 0.5 | 0.3259 | — | |
| type of residue % waxy 5 MIN | 34.5 | 31.8 | 0.2622 | — | |
| type of residue %-greasy 5 MIN | 9.5 B | 26.5 A | <.0001 | 5.12 | ** |
| type of residue %-silicone 5 MIN | 23.3 | 23.3 | 1.0000 | — | |
| type of residue %- | 32.8 A | 18.0 B | <.0001 | 6.03 | ** |
| powdery/chalky 5 MIN | | | | | |
| type of residue %-plastic 5 MIN | 0.0 | 0.0 | — | — | |
| type of residue-oily 5 MIN | 0.0 | 0.0 | 0.3259 | — | |
| type of residue-waxy 5 MIN | 2.8 | 2.7 | 0.8114 | — | |
| type of residue-greasy 5 MIN | 0.8 B | 2.4 A | <.0001 | 0.54 | ** |
| type of residue-silicone 5 MIN | 1.9 | 2.1 | 0.3876 | — | |
| type of residue- | 2.5 A | 1.4 B | <.0001 | 0.47 | ** |
| powdery/chalky 5 MIN | | | | | |
| type of residue-plastic 5 MIN | 0.0 | 0.0 | — | — | |
| stickiness, crease 5 MIN | 0.1 | 0.1 | 1.0000 | — | |
| gloss, skin 15 MIN | 8.5 B | 9.2 A | 0.0257 | 0.56 | ** |
| whitening 15 MIN | 0.3 a | 0.0 b | 0.0887 | 0.29 | * |
| wetness 15 MIN | 5.3 | 5.4 | 0.6770 | — | |
| stickiness, skin 15 MIN | 0.0 | 0.0 | — | — | |
| slipperiness 15 MIN | 81.2 b | 81.9 a | 0.0878 | 0.63 | * |
| thickness of residue 15 MIN | 4.3 B | 5.1 A | 0.0065 | 0.59 | ** |
| amount of residue 15 MIN | 6.0 | 6.5 | 0.1241 | — | |
| type of residue %-oily 15 MIN | 0.0 | 0.0 | — | — | |
| type of residue % waxy 15 MIN | 37.0 | 34.3 | 0.3156 | — | |
| type of residue %-greasy | 4.5 B | 19.8 A | <.0001 | 4.97 | ** |
| 15 MIN | | | | | |
| type of residue %-silicone | 23.0 | 22.0 | 0.6274 | — | |
| 15 MIN | | | | | |
| type of residue %- | 35.5 A | 24.0 B | 0.0007 | 6.18 | ** |
| powdery/chalky 15 MIN | | | | | |
| type of residue %-plastic | 0.0 | 0.0 | — | — | |
| 15 MIN | | | | | |
| type of residue-oily 15 MIN | 0.0 | 0.0 | — | — | |
| type of residue-waxy 15 MIN | 2.2 | 2.3 | 0.7539 | — | |
| type of residue-greasy 15 MIN | 0.2 B | 1.3 A | <.0001 | 0.43 | ** |
| type of residue-silicone 15 MIN | 1.4 | 1.5 | 0.8264 | — | |
| type of residue- | 2.1 A | 1.4 B | 0.0005 | 0.35 | ** |
| powdery/chalky 15 MIN | | | | | |
| type of residue-plastic 15 MIN | 0.0 | 0.0 | — | — | |
| stickiness, crease 15 MIN | 0.0 b | 0.1 a | 0.0887 | 0.10 | * |
| tautness 15 MIN | 15.2 A | 14.9 B | 0.0432 | 0.24 | ** |
| rub-off whitening 15 MIN | 8.9 A | 3.7 B | <.0001 | 1.86 | ** |

### Example 2: Subject Satisfaction Survey

Fifty subjects were selected to use and compare an antiperspirant containing cyclopentasiloxane (D5) with an identical antiperspirant where hemisqualane replaced the cyclopentasiloxane (D5). The subjects were asked to rate their satisfaction of each antiperspirant among fifteen product aspects: flakiness, greasiness, stickiness, softness, hardness, ease of application, ease of spreading, evenness of application, greasy feeling, skin dryness, lightness on skin, absorbs into skin, dry skin feeling, residue, and willingness to purchase product.

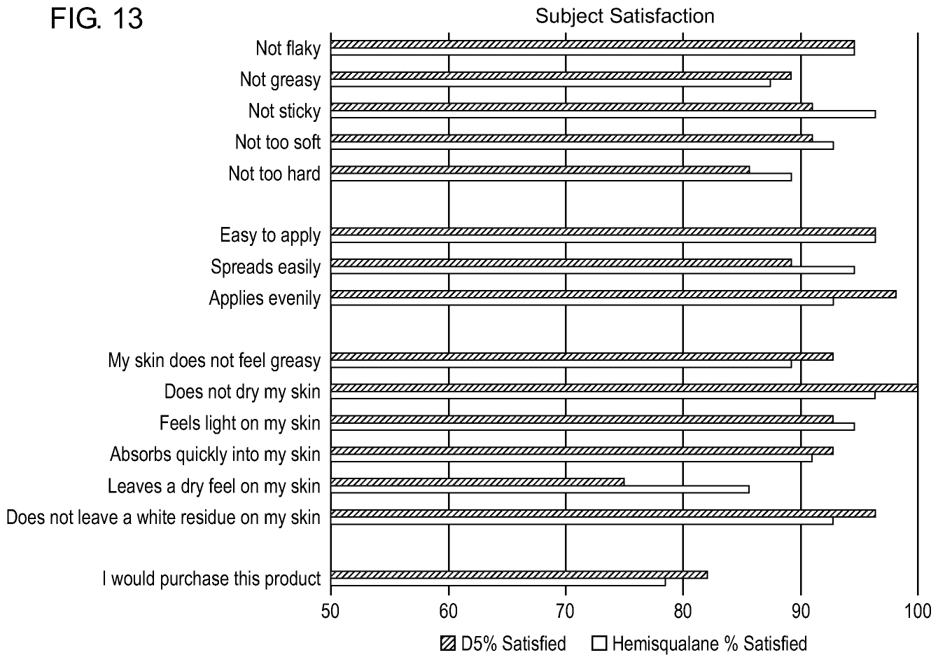

As shown in FIG. 13, the two antiperspirant formulations scored similarly across the fifteen aspects. This data demonstrates that hemisqualane based antiperspirants are satisfactory substitutions for cyclopentasiloxane (D5) based antiperspirants.

1. An antiperspirant formulation comprising hemisqualane.

2. The antiperspirant formulation of claim 1, further comprising one or more solid dry-feel particles.

3. The antiperspirant formulation of claim 2, wherein the solid dry-feel particle is a solid absorbent particle.

4. The antiperspirant formulation of claim 2, wherein the solid dry-feel particle is a solid adsorbent particle.

5. The antiperspirant formulation of claim 1, wherein the antiperspirant is not an aerosol.

6. The antiperspirant formulation of claim 1, wherein the formulation does not contain an organosilicon compound.

7. The antiperspirant formulation of claim 6, wherein the organosilicon compound is a cyclomethicone.

8. The antiperspirant formulation of claim 7, wherein the cyclomethicone is cyclotetrasiloxane (D4).

9. The antiperspirant formulation of claim 7, wherein the cyclomethicone is cyclopentasiloxane (D5).

10. The antiperspirant formulation of claim 7, wherein the cyclomethicone is cyclohexasiloxane (D6).

11. The antiperspirant formulation of claim 4, wherein the solid adsorbent particle comprises from about 0% to about 5% of the formulation.

12. The antiperspirant formulation of claim 4, wherein the one or more solid adsorbent particle is selected from polymer-based microspheres, allyl methacrylates crosspolymers, lauryl methacrylate/glycol dimethacrylate crosspolymer, silica, and talc.

13. The antiperspirant formulation of claim 1 further comprising an active ingredient.

14. The antiperspirant formulation of claim 13, wherein the active ingredient comprises an aluminum salt.

15. The antiperspirant formulation of claim 13, wherein the active ingredient is selected from aluminum chloride; aluminum chlorohydrate; aluminum chlorodydrex; aluminum chlorohydrex propylene glycol; aluminum dichlorodhydrate; aluminum dichlorodhydrex polyethylene glycol; aluminum dichlorohydrex propylene glycol; aluminum sesquichlorohydrate; aluminum sesquichlorohydrex propylene glycol; aluminum zirconium octachlorohydrate; aluminum zirconium octachlorohydrex gly.; aluminum zirconium pentachlorohydrate; aluminum zirconium pentachlorohydrex gly; aluminum zirconium tetrachlorohydrate; aluminum zirconium tetrachlorohydrex gly; aluminum zirconium trichlorohydrate; and aluminum zirconium trichlorohydrex gly.

16. The antiperspirant formulation of claim 15, wherein the active ingredient is aluminum zirconium tetrachlorohydrex glycine.

17. The antiperspirant formulation claim 13, wherein the active ingredient comprises from about 0.1% to about 30% (w/w).

18. The antiperspirant formulation of claim 17, wherein the active ingredient comprises from about 10% to about 25% (w/w).

19. The antiperspirant formulation of claim 18, wherein the active ingredient comprises from about 16% to about 20%.

20. The antiperspirant formulation of claim 19, wherein the active ingredient comprises about 18% (w/w).

21. The antiperspirant formulation of claim 1 further comprising one or more inactive ingredients.

22. The antiperspirant formulation of claim 21, wherein the one or more inactive ingredients are selected from wax, emulsifier, emollient, moisturizer, anti-bacterial agent, preservative, humectant, buffer, color, and fragrance.

23. The antiperspirant formulation of claim 1, wherein hemisqualane comprises from about 1% to about 80% (w/w) of the antiperspirant formulation.

24. The antiperspirant formulation of claim 23, wherein the hemisqualane comprises from about 25% to about 75% (w/w) of the antiperspirant formulation.

25. A method of preventing perspiration comprising applying an antiperspirant comprising the antiperspirant formulation of claim 1 to an area of skin in need of application.

26. The method of claim 25, wherein the antiperspirant is applied at least once every 24 hours.

27. The method of claim 25, wherein the antiperspirant is applied only once every 24 hours.
